(19) United States
(12) Patent Application Publication
(10) Pub. No.: US 2024/0001062 A1
(43) Pub. Date: Jan. 4, 2024

(54) METHOD AND SYSTEM FOR PATIENT INTERFACE DEVICE DATA COLLECTION WITH PRIVACY

(71) Applicant: KONINKLIJKE PHILIPS N.V., Eindhoven (NL)

(72) Inventors: DMITRY NIKOLAEVICH ZNAMENSKIY, EINDHOVEN (NL); PRAVEEN KUMAR PANDIAN SHANMUGANATHAN, MONROEVILLE, PA (US)

(21) Appl. No.: 18/214,607

(22) Filed: Jun. 27, 2023

Related U.S. Application Data
(60) Provisional application No. 63/356,647, filed on Jun. 29, 2022.

(51) Int. Cl. A61M 16/06; G06T 17/20

(52) U.S. Cl. CPC A61M 16/06; G06T 17/205; G06T 2207/30201; A61M 2016/0661

(57) ABSTRACT

A 3D data collection method with privacy protection includes obtaining a 3D scan of at least a portion of a patient, receiving an avatar template and control point registration data, identifying control points on the 3D scan based on the control point registration data, adjusting the avatar template to coincide control points on the avatar template with control points on the 3D scan, and transmitting the adjusted avatar template.

### CROSS-REFERENCE TO RELATED APPLICATIONS

This patent application claims the priority benefit under 35 U.S.C. § 119(e) of U.S. Provisional Application No. 63/356,647, filed on Jun. 29, 2022, the contents of which are herein incorporated by reference.

### BACKGROUND OF THE INVENTION

### 1. Field of the Invention

The disclosed concept pertains to data collection for patient interface device selection, creation, or customization, and, in particular, to data collection systems and method for patient interface device selection, creation, or customization that retain private and proprietary information.

### 2. Description of the Related Art

Many individuals suffer from disordered breathing during sleep. Sleep apnea is a common example of such sleep disordered breathing suffered by millions of people throughout the world. One type of sleep apnea is obstructive sleep apnea (OSA), which is a condition in which sleep is repeatedly interrupted by an inability to breathe due to an obstruction of the airway; typically the upper airway or pharyngeal area. Obstruction of the airway is generally believed to be due, at least in part, to a general relaxation of the muscles which stabilize the upper airway segment, thereby allowing the tissues to collapse the airway. Another type of sleep apnea syndrome is a central apnea, which is a cessation of respiration due to the absence of respiratory signals from the brain's respiratory center. An apnea condition, whether OSA, central, or mixed, which is a combination of OSA and central, is defined as the complete or near cessation of breathing, for example a 90% or greater reduction in peak respiratory air-flow.

Those afflicted with sleep apnea experience sleep fragmentation and complete or nearly complete cessation of ventilation intermittently during sleep with potentially severe degrees of oxyhemoglobin desaturation. These symptoms may be translated clinically into extreme daytime sleepiness, cardiac arrhythmias, pulmonary-artery hypertension, congestive heart failure and/or cognitive dysfunction. Other consequences of sleep apnea include right ventricular dysfunction, carbon dioxide retention during wakefulness, as well as during sleep, and continuous reduced arterial oxygen tension. Sleep apnea sufferers may be at risk for excessive mortality from these factors as well as by an elevated risk for accidents while driving and/or operating potentially dangerous equipment.

Even if a patient does not suffer from a complete or nearly complete obstruction of the airway, it is also known that adverse effects, such as arousals from sleep, can occur where there is only a partial obstruction of the airway. Partial obstruction of the airway typically results in shallow breathing referred to as a hypopnea. A hypopnea is typically defined as a 50% or greater reduction in the peak respiratory air-flow followed by oxyhemoglobin desaturation and/or a cortical arousal. Other types of sleep disordered breathing include, without limitation, upper airway resistance syndrome (UARS) and vibration of the airway, such as vibration of the pharyngeal wall, commonly referred to as snoring.

It is well known to treat sleep disordered breathing by applying a positive airway pressure (PAP) to the patient's airway using an airway pressure support system that typically includes a mask, a pressure generating device, and a conduit to deliver positive pressure breathing gas from the pressure generating device to the patient through the mask. This positive pressure effectively “splints” the airway, thereby maintaining an open passage to the lungs. In one type of PAP therapy, known as continuous positive airway pressure (CPAP), the pressure of gas delivered to the patient is constant throughout the patient's breathing cycle. It is also known to provide a positive pressure therapy in which the pressure of gas delivered to the patient varies with the patient's breathing cycle, or varies with the patient's effort, to increase the comfort to the patient. This pressure support technique is referred to as bi-level pressure support, in which the inspiratory positive airway pressure (IPAP) delivered to the patient is higher than the expiratory positive airway pressure (EPAP). It is further known to provide a positive pressure therapy in which the pressure is automatically adjusted based on the detected conditions of the patient, such as whether the patient is experiencing an apnea and/or hypopnea. This pressure support technique is referred to as an auto-titration type of pressure support, because the pressure support device seeks to provide a pressure to the patient that is only as high as necessary to treat the disordered breathing.

Pressure support therapies as just described involve the placement of a patient interface device including a mask component having a soft, flexible sealing cushion on the face of the patient. The mask component may be, without limitation, a nasal mask that covers the patient's nose, a nasal/oral mask that covers the patient's nose and mouth, or a full face mask that covers the patient's face. Such patient interface devices may also employ other patient contacting components, such as forehead supports, cheek pads and chin pads. The patient interface device is typically secured to the patient's head by a headgear component. The patient interface device is connected to a gas delivery tube or conduit and interfaces the pressure support device with the airway of the patient, so that a flow of breathing gas can be delivered from the pressure/flow generating device to the airway of the patient.

It is important that the patient interface device fits the patient. An improper fit can lead to a patient interface device that does not properly seal against the patient's face, which can cause to leaks that are detrimental to the patient's pressure support therapy. Additionally, a properly fitting patient interface device should be comfortable for the patient to wear. A patient interface device that does not fit properly could cause discomfort due to being too tight, rubbing against certain points on the patient's face, or other uncomfortable symptoms of an improper fit. The discomfort can lead to the patient choosing to discontinue pressure support therapy.

Some methods that assist with selection of a properly fitting patient interface device include performing a 3D scan of the patient's face, and subsequent geometric analysis of the 3D scan to assist with selection of a properly fitting patient interface device. The 3D scan is often conducted by a party different than that analyzing the 3D scan, thus necessitating transmittal of the 3D scan. The 3D scan contains personal information that the patient would prefer to remain private and not be transmitted to different parties. Thus, there remains room for improvement in data collection associated with patient interface device selection.

### SUMMARY OF THE INVENTION

In accordance with an aspect of the disclosed concept, a 3D data collection method with privacy protection comprises: obtaining a 3D scan of at least a portion of a patient at a local site; receiving an avatar template and control point registration data from an external site; identifying control points on the 3D scan based on the control point registration data; adjusting the avatar template to coincide control points on the avatar template with control points on the 3D scan; and transmitting the adjusted avatar template to the external site.

In accordance with an aspect of the disclosed concept, a system for data collection with privacy comprises: a scanning unit structured to obtain a 3D scan of at least a portion of a patient at a local site; a memory structured to store one or more routines; a processing unit structured to execute the one or more routines, wherein execution of the one or more routines causes the processor to: receive an avatar template and control point registration data from an external site; identify control points on the 3D scan based on the control point registration data; adjust the avatar template to coincide control points on the avatar template with control points on the 3D scan; and transmit the adjusted avatar template to the external site.

In accordance with an aspect of the disclosed concept, a non-transitory computer readable medium storing one or more programs, including instructions, which when executed by a computer, causes the computer to perform a method of 3D data collection with privacy protection. The method comprises: obtaining a 3D scan of at least a portion of a patient at a local site; receiving an avatar template and control point registration data from an external site; identifying control points on the 3D scan based on the control point registration data; adjusting the avatar template to coincide control points on the avatar template with control points on the 3D scan; and transmitting the adjusted avatar template to the external site.

### BRIEF DESCRIPTION OF THE DRAWINGS

FIG. 1 is a flowchart of a method of data collection by a provider according to an exemplary embodiment of the disclosed concept;

FIG. 2 is a flowchart of a method of data collection by a data collection site according to an exemplary embodiment of the disclosed concept;

FIG. 3 is a flowchart of a method of data collection by a provider and a data collection site according to an exemplary embodiment of the disclosed concept;

FIG. 4 is an image of an average 3D head template with control points according to an exemplary embodiment of the disclosed concept;

FIG. 5 is an image of an avatar template with control points according to an exemplary embodiment of the disclosed concept;

FIG. 6 is an image of an average 3D head template adjusted with 3D scan data;

FIG. 7 is an image of an adjusted avatar template according to an exemplary embodiment of the disclosed concept; and

FIG. 8 is a schematic diagram of a system for data collection with privacy according to an exemplary embodiment of the disclosed concept.

### DETAILED DESCRIPTION OF EXEMPLARY EMBODIMENTS

As used herein, the singular form of “a”, “an”, and “the” include plural references unless the context clearly dictates otherwise.

The disclosed concept relates to systems and methods for data collection that protect privacy and proprietary information. Example embodiments of the disclosed concept will be described with respect to data collection for the purposes of selecting or customizing a patient interface device (e.g., a mask) for respiratory therapy. However, it will be appreciated that the disclosed concept is also pertinent to other applications where private data is collected and potentially transmitted to other parties.

In one embodiment, an avatar template (e.g., a blurred 3D average head) and control points are transmitted from a provider to a data collection site. At the data collection site, a 3D scan of a patient is obtained and control points on the 3D scan are identified. The avatar template is adjusted to coincide control points on the avatar template with control point on the 3D scan of the patient. The adjusted avatar template is then transmitted back to the provider where the adjusted control points are retrieved. The provider may use the adjusted control points to select or customize a patient interface device for the patient, or retain the adjusted control points for general development of better fitting patient interface devices or other purposes. From the adjusted avatar template, it is not reasonably possible to identify the patient. Additionally, from the adjusted avatar template, it is not reasonably possible to discern any proprietary data or algorithms the provider may use for determining patient interface device selection or customization. Some example embodiments of the disclosed concept will be described in more detail herein.

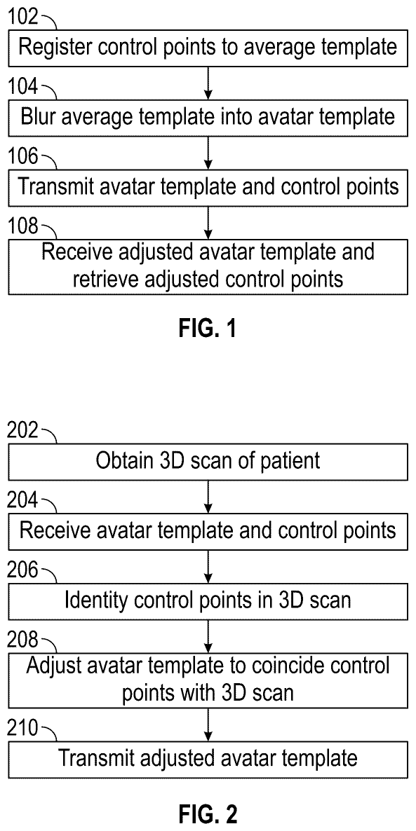

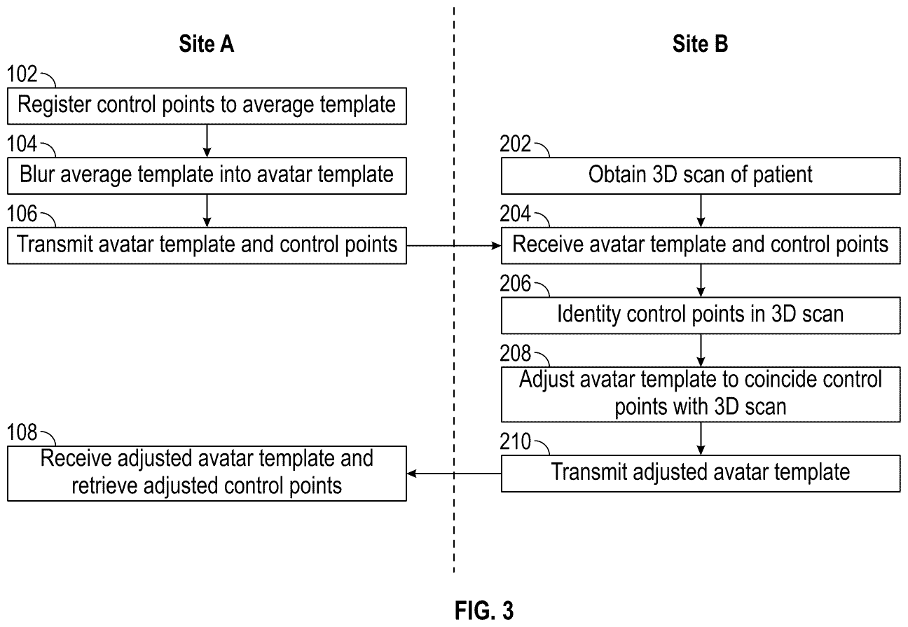

FIG. 1 is a flowchart of a method of data collection performed by a provider and FIG. 2 is a flowchart of a method of data collection performed at a data collection site. FIG. 3 is a combined flowchart of a method of data collection performed by both the provider and the data collection site. Referring to FIG. 1, the method begins at 102 where control points are registered to an average 3D head template. The average 3D head template is a 3D model of an average head not associated with any particular patient. The control points may be 3D points relevant to a particular product or design. For example, in the case of a patient interface device, the control points may be associated with contours relevant to determining the fit of a patient interface to a patient's face. For example, the control points may be located along contours around the nose, mouth, and cheeks. Registering the control points to the average 3D head template includes associating the control points with points on the average 3D head template. For example, the control points may be registered such that the location of the control points can be computed from vertices of the template. As an example, the equation C=P*V, where C is the control points, V is the template vertices, and P is a sparse registration matrix may be used. Using the above relation, updated locations of the control points can be retrieved from the template after the template is adjusted.

At 104, the average 3D head template is blurred into an avatar template. The avatar template is an excessively smoothed average of the average 3D head template so that when the avatar template is subsequently adjusted based on the 3D scan of a patient, no facial detail from the patient's 3D scan will appear on the adjusted avatar template. The avatar template may be generated, for example, using the local averaging kernel applied to a regular average 3D head template, followed by surface reparameterization. The surface reparameterization creates an efficient technical barrier for the non-authorized analysis of adjusted avatar templates by a third party in the case that an unauthorized third party were to obtain an adjusted avatar template. The reparameterization may be implemented as a random vertex displacement followed by a quadric edge collapse decimation.

At 106, the avatar template and control point registration data (e.g., the sparse registration matrix) is transmitted to, for example, a data collection site. In an embodiment, the transfer is performed is encrypted, as the control point registration data may be considered proprietary by the provider.

After the avatar template and control point registration data is transmitted to the data collection site, the data collection site will generate an adjusted avatar template in which the control points on the avatar template are adjusted to coincide with control points of a 3D scan of the patient, as will be described in more detail with respect to FIG. 2. At the provider, at 108, the adjusted avatar template is received from the data collection site. From the adjusted avatar template, the provide retrieves the adjusted control points. For example, using the sparse registration matrix and the adjusted avatar template, the provide is able to use the relation C=P*V described above, to retrieve the adjusted control points. The control points may then be used to determine contours and selection or customization of a patient interface device, or for other purposes by the provider.

Referring to FIG. 2, data collection action performed at the data collection site will be described. The data collection site may refer to any site where a 3D scan of a patient is performed. The data collection site may be, for example, a health services site or may be another site where the patient is located. At 202, a 3D scan of a patient is obtained. The 3D scan of the patient may be obtained, for example, using a 3-D optical scanner, a camera, a push-pin array, or any other device suitable for creating a 3D model of the patient's face. The 3D scan may be obtained for only a relevant portion of the patient. For example, in the case of fitting a patient interface device, the 3D scan may be of the patient's head. For other applications, other parts of the patient may be scanned.

At 204, the avatar template and control points registration data are received from the provider. As described above, the avatar template and control points registration data were transmitted at 106 in FIG. 1.

At 206, control points are identified in the 3D scan of the patient. To identify the control point in the 3D scan of the patient, in one embodiment, a two part process may be performed. As the first part, the avatar template is morphed to fit the 3D scan of the patient. For example, landmarks (e.g., defining features such as the tip of the nose, edges of the mouth, etc.) may be used to roughly morph the avatar template to the 3D scan of the patient. This morphing primarily aligns the avatar template with the 3D scan of the patient and does not result in identifiable characteristics of the 3D scan of the patient transferring to the avatar template. As a second part of the process, the control points of the avatar template are projected onto the 3D scan of the patient, thus identifying the control points on the 3D scan of the patient.

At 208, the avatar template is adjusted such that the control points on the avatar template coincide with the control points on the 3D scan of the patient. In an embodiment, the avatar template is morphed such that the control points on the avatar template coincide with the control points on the 3D scan of the patient. That is, the control points on the adjusted avatar template with generate the same contours as the control points on the 3D scan of the patient. In an embodiment, Laplacian mesh editing, such as that described in O. Sorkine et al. Laplacian mesh editing. Eurographics Symposium on Geometry Processing (2004), which is hereby incorporated by reference in its entirety, may be used to morph the avatar template. However, it will be appreciated that other processes may be used without departing from the scope of the disclosed concept.

At 210, the adjusted avatar template is transmitted to the provider. The adjusted avatar template may be transmitted in an open manner as the adjusted avatar template does not include any information that would identify the patient, nor does it include any proprietary information. In some embodiments, the adjusted avatar template may be shown to the patient, and the patient may provide confirmation of permission to transmit the adjusted avatar template to the provider. As discussed above with respect to FIG. 1, the provider receives the adjusted avatar template at 108 and may then retrieve the adjusted control points and use them for patient interface device selection or customization, or other purposes.

Referring to FIG. 3, the flowcharts of FIGS. 1 and 2 are combined to show the entire data collection process. In FIG. 3, the left side of the flowchart is performed at SITE A, which may be a provider, and the right side of the flowchart is performed at SITE B, which may be a data collection site.

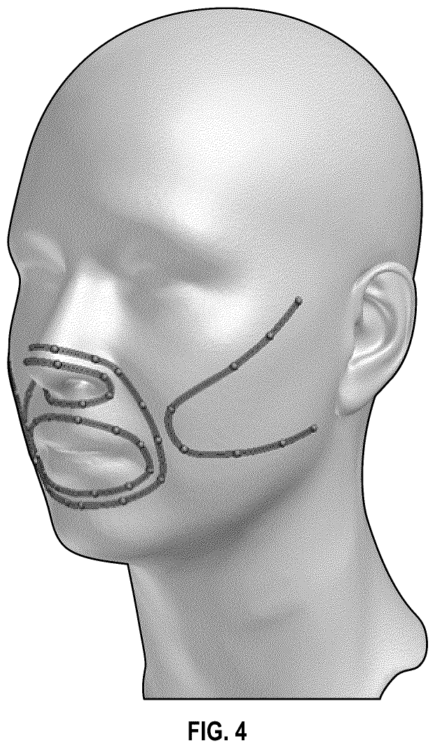

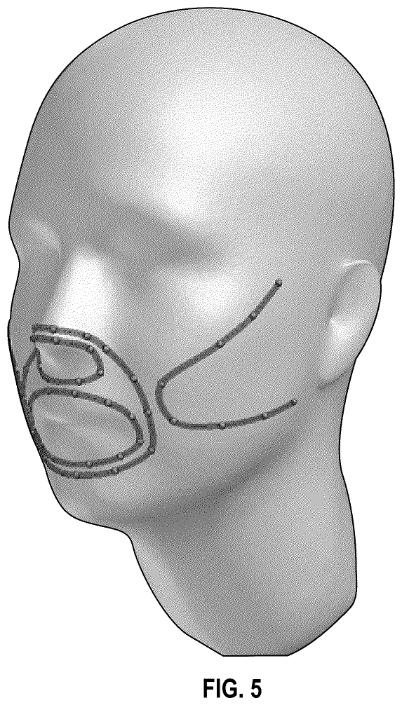

FIGS. 4-7 include images of various 3D models. FIG. 4 is an average 3D head template including control points and derived contours which can be used for mask design and/or selection purposes. The average 3D head template and control points are an example of those used at 102 described above with respect to FIG. 1. FIG. 5 is an avatar template including control points which can be used for mask design and/or selection purposes, derived from the average 3D head template of FIG. 4. As shown in FIG. 5, the avatar template is blurred and includes less identifiable features compared to the average 3D head template of FIG. 4.

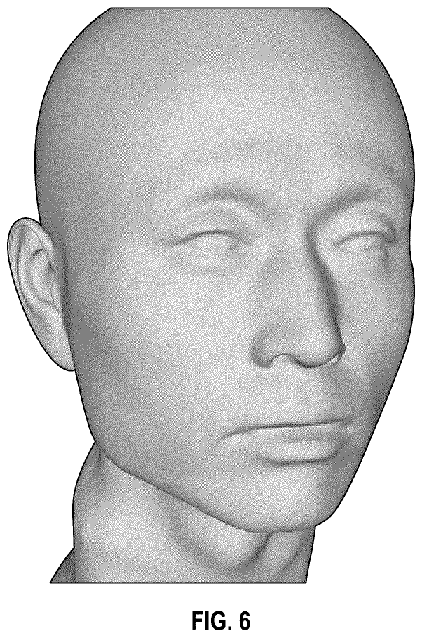

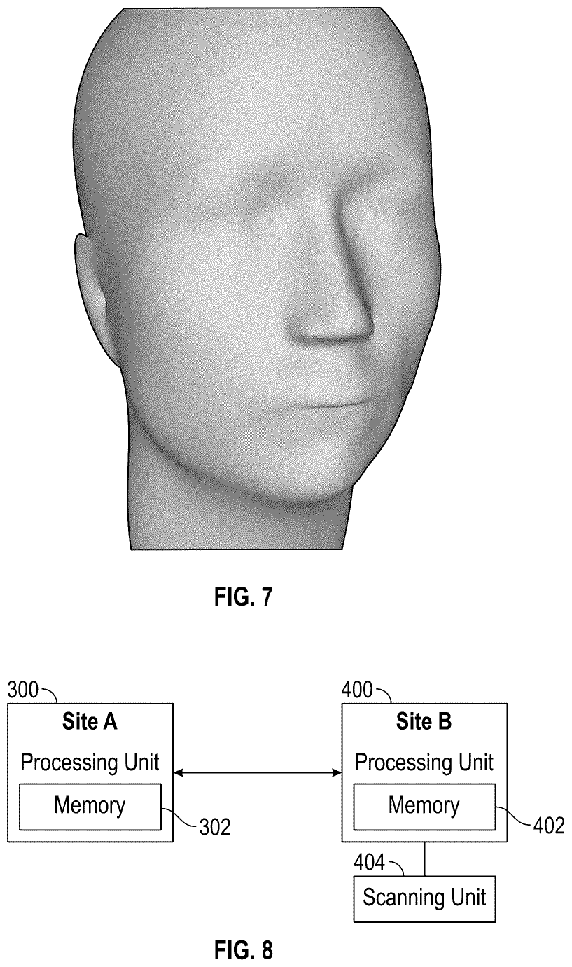

FIG. 6 is an image of an average 3D head template modified based on a 3D scan of a patient. As shown in FIG. 6, the image is somewhat recognizable. That is, although the model is an average head template, adjusting it to features of a patient may make the result recognizable as the user. This image is not used in the process. FIG. 7 is an image of an adjusted avatar template which is used in the process. As shown in FIG. 7, when the avatar template is adjusted based on the 3D scan of the patient, the resultant adjusted avatar template is unrecognizable and presents not risk of the patient being identified based on the model. That is, even if the adjusted avatar template were obtained by an unauthorized third party, it could not be used to identify the patient.

FIG. 8 is a schematic diagram of a system that may be used to implement the methods described with respect to FIGS. 1-3. The system includes a SITE A processing unit 300 and a SITE B processing unit 400. The SITE A processing unit 300 may be part of the provider and the SITE B processing unit 400 may be part of the data collection site. The processing units 300,400 may be any type of processing device including a processor, such as a computer, laptop, tablet, mobile phone etc. The processing units 300,400 may include a memory 302,402 such as any non-transitory computer readable medium. The memory 302,402 may store one or more routines which when executed by the corresponding processor, may cause the processor to implement one or more steps of the methods shown and described with respect to FIGS. 1-3. In an embodiment, the a scanning unit 404 structured to obtain a 3D scan of a patient may be associated with the SITE B processing unit 400.

It is contemplated that aspects of the disclosed concept can be embodied as computer readable codes on a tangible computer readable recording medium. The computer readable recording medium is any data storage device that can store data which can be thereafter read by a computer system. Examples of the computer readable recording medium include read-only memory (ROM), random-access memory (RAM), CD-ROMs, magnetic tapes, floppy disks, and optical data storage devices.

In the claims, any reference signs placed between parentheses shall not be construed as limiting the claim. The word “comprising” or “including” does not exclude the presence of elements or steps other than those listed in a claim. In a device claim enumerating several means, several of these means may be embodied by one and the same item of hardware. The word “a” or “an” preceding an element does not exclude the presence of a plurality of such elements. In any device claim enumerating several means, several of these means may be embodied by one and the same item of hardware. The mere fact that certain elements are recited in mutually different dependent claims does not indicate that these elements cannot be used in combination.

Although the invention has been described in detail for the purpose of illustration based on what is currently considered to be the most practical and preferred embodiments, it is to be understood that such detail is solely for that purpose and that the invention is not limited to the disclosed embodiments, but, on the contrary, is intended to cover modifications and equivalent arrangements that are within the spirit and scope of the appended claims. For example, it is to be understood that the present invention contemplates that, to the extent possible, one or more features of any embodiment can be combined with one or more features of any other embodiment.

What is claimed is:

1. A 3D data collection method with privacy protection, the method comprising:
obtaining a 3D scan of at least a portion of a patient at a local site;
receiving an avatar template and control point registration data from an external site;
identifying control points on the 3D scan based on the control point registration data;
adjusting the avatar template to coincide control points on the avatar template with control points on the 3D scan; and
transmitting the adjusted avatar template to the external site.

2. The method of claim 1, further comprising:
generating the avatar template and control point registration data at the external site; and
transmitting the avatar template and control point registration data to the local site.

3. The method of claim 2, wherein generating the avatar template includes:
registering control points to an average template; and
blurring the average template to generate the avatar template.

4. The method of claim 3, wherein blurring the average template includes at least one of random vertex displacement and decimation of the average template.

5. The method of claim 1, wherein the control point registration data associates control points with the avatar template.

6. The method of claim 5, wherein the control point registration data includes a sparse registration matrix.

7. The method of claim 1, wherein identifying (206) control points on the 3D scan based on the control point registration data includes:
morphing the avatar template to fit the 3D scan; and
projecting control points of the avatar template onto the 3D scan.

8. The method of claim 7, wherein morphing the avatar template to fit the 3D scan includes matching landmarks of the avatar template and the 3D scan.

9. The method of claim 1, wherein adjusting the avatar template to coincide control points on the avatar template with control points on the 3D scan includes morphing the avatar template using Laplacian mesh editing.

10. The method of claim 1, further comprising retrieving adjusted control points from the adjusted avatar template.

11. The method of claim 10, further comprising using the adjusted control points to select or customize a patient interface device for the patient.

12. The method of claim 1, wherein the 3D scan includes a scan of the patient's face, and wherein the patient is not identifiable from the adjusted avatar template.

13. The method of claim 1, wherein the avatar template and control point registration data is received in an encrypted communication and the adjusted avatar template is transmitted in an open communication.

14. A system for data collection with privacy, the system comprising:
a scanning unit structured to obtain a 3D scan of at least a portion of a patient at a local site;
a memory structured to store one or more routines;
a processing unit structured to execute the one or more routines, wherein execution of the one or more routines causes the processor to:
receive an avatar template and control point registration data from an external site;
identify control points on the 3D scan based on the control point registration data;
adjust the avatar template to coincide control points on the avatar template with control points on the 3D scan; and
transmit the adjusted avatar template to the external site.

15. A non-transitory computer readable medium storing one or more programs, including instructions, which when executed by a computer, causes the computer to perform a method of 3D data collection with privacy protection, the method comprising:
obtaining a 3D scan of at least a portion of a patient at a local site;
receiving an avatar template and control point registration data from an external site;
identifying control points on the 3D scan based on the control point registration data;
adjusting the avatar template to coincide control points on the avatar template with control points on the 3D scan; and
transmitting the adjusted avatar template to the external site.
